(19) United States
(12) Patent Application Publication
(10) Pub. No.: US 2001/0036644 A1
(43) Pub. Date: Nov. 1, 2001

(54) Autoimmune inner ear disease diagnostic assay

(76) Inventor: Tai-June Yoo, Memphis, TN (US)

Correspondence Address: Jamie L. Greene, KILPATRICK STOCKTON LLP, 2400 Monarch Tower, 3424 Peachtree Road, N.E., Atlanta, GA, 30326, US

(21) Appl. No.: 09/790,984

(22) Filed: Feb. 22, 2001

Related U.S. Application Data
(60) Provisional application No. 60/184,085, filed on Feb. 22, 2000.
(60) Provisional application No. 60/184,141, filed on Feb. 22, 2000.

(51) Int. Cl. G01N 33/53; G01N 33/537; G01N 33/543

(52) U.S. Cl. 435/7.92

(57) ABSTRACT

Methods for diagnosing and monitoring autoimmune inner ear diseases such as Meniere's disease by combining a sample with one or more antigens and detecting the binding of antibodies in the sample to the antigen. The antigen is an inner ear collagen protein or peptide including type II collagen, type IX collagen, or type XI collagen. Binding is detected using an assay method such as an ELISA immunoassay. The assay may further include one or more additional inner ear antigens such as the signal transduction protein Raf-1, the myelin protein P0 and β-tubulin.

This application claims priority to Provisional Patent Application No. 60/184,085 filed Feb. 22, 2000 and Provisional Patent Application No. 60/184,141 filed Feb. 22, 2000.

### FIELD OF THE INVENTION

This relates to the field of immunology and more specifically relates to immunoassay methods for detecting an autoimmune disease of the inner ear.

### BACKGROUND OF THE INVENTION

Hearing problems can result from a variety of disorders, diseases or traumas of the inner ear. Symptoms of inner ear problems include, but are not limited to, hearing loss, dizziness, vertigo and tinnitus. Several inner ear diseases have recently been classified as autoimmune diseases. These include, but are not limited to, Meniere's disease, progressive bilateral sensorineural hearing loss (PSHL), otosclerosis and sudden hearing loss.

Meniere's disease is an idiopathic inner ear disease with the triad symptoms of fluctuating hearing loss, episodic vertigo, and tinnitus. These symptoms may be produced by a sudden influx of fluid into the endolymphatic sac, resulting in a rupture of Reisser's membrane in the cochlea. Immunological derangement of the endolymphatic sac or other membranous structures of the inner ear may initiate a cascade of reactions leading to endolymphatic hydrops and presenting as Meniere's disease.

There are at least four million Meniere's disease patients in the United States, and many more patients report symptoms associated with Meniere's disease but cannot be positively diagnosed.

Although idiopathic by definition, Meniere's disease has been ascribed to a variety of causes, among which are autoimmune factors. The immune mechanisms of Meniere's disease, have yet to be fully defined. The weight of recent evidence strongly implicates immunologic and/or chronic inflammatory processes as contributing to the pathogenesis of this illness. Several key factors, such as T_{H} cells, B cells, MHC, antigen, cytokines etc., contribute to the immune response in inner ear diseases. There is evidence to suggest that antibodies generated against inner ear proteins cause inner ear inflammation and swelling that can result in a complete loss of hearing.

Researchers have attempted to isolate the antigen or antigens responsible for autoimmune inner ear diseases, but have achieved little success.

A protein is considered to be a potential antigen if it is reactive with antibodies produced by patients exhibiting autoimmune inner ear diseases. For example, antibodies in the sera of patients having inner ear disease have been found to react with protein bands of 58 kD and of 30 kD on Western blots of guinea pig inner ear extracts (Cao, M. et al., Laryngoscope 106:207-212, 1996). The 58 kD band was shown to be nonspecific to the inner ear when antibodies reacted with a 58 kD band on Western blots of guinea pig brain, lung and liver. In contrast, the 30 kD band was specific to the inner ear. Antibodies from patients reacted with a 30 kD band on Western blots of extracts from Corti's organ, the spiral ganglion and the acoustic nerve fiber, but not with extracts from the spinal ligament and the stria vascularis.

Antibodies against a 30 kD cochlear protein have been in reported in the serum of some patients with Meniere's disease (Joliat, T. et al., Ann. Otol. Rhinol. Laryngol. 101:1001-1006, 1994 and Cao, M. et al., Laryngoscope 106:207-212, 1996). This 30 kD protein has been identified as the major peripheral myelin protein “P0” and is believed to be associated with acoustic nerve and spiral ganglion (Cao, M. et al., Laryngoscope 106:207-212 (1996). However, antibodies reactive with the 30 kD protein are not specific for Meniere's disease because these antibodies have been found in patients having other autoimmune diseases such as progressive bilateral sensorineural hearing loss (PSNHL), otosclerosis, sudden deafness and in control subjects Antibodies against a 68 kD protein in extracts from bovine inner ear have been reported in the serum of PSNHL patients (Harris J. and Sharp P. Laryngoscope 100:516-524, 1990). However, this 68 kD protein is not specific to Meniere's disease because it has been identified as a 70 kD heat shock protein that has been implicated in other autoimmune diseases such as Lyme's disease and ulcerative colitis.

An early diagnosis of autoimmune inner ear disease is critical. Prompt treatment of the disease at an early stage of the illness may preserve any remaining inner ear function. Moreover, the ability to distinguish antigenic epitopes of the inner ear relevant to the pathogenesis of specific autoimmune inner ear diseases will enable clinical investigation and research on autoimmune inner ear disease, and will further enable the clinical diagnosis of autoimmune inner ear diseases and immunologic therapy.

As the availability of human inner ear tissue is extremely limited, there is an on-going need for the identification of disease-specific antigens and for the development of simple, sensitive and reproducible assays for the detection and differential diagnosis of autoimmune inner ear diseases.

### SUMMARY OF THE INVENTION

Methods for diagnosing and monitoring autoimmune inner ear diseases such as Meniere's disease are provided. The detection of type II, type IX, or type XI collagen antibodies, or a combination thereof, in a sample from a patient indicates that the patient suffers from an autoimmune inner ear disease such as Meniere's disease.

In accordance with the assay method a sample is combined with one or more antigens, and the binding of antibodies in the sample to the antigens is detected. Preferably, the antigens are from the membranous structures of the inner ear. The antigens are inner ear proteins identified herein as including type II collagen protein or an antigenic fragment or peptide thereof, type IX collagen protein or an antigenic fragment or peptide thereof, type XI collagen protein or an antigenic fragment or peptide thereof, or a combination of these proteins or fragments. The binding of antibodies in the sample to one or more of the collagen proteins or peptides is detected using methods well known to those skilled in the art, such as an immunoassay, preferably an ELISA immunoassay.

In a preferred method, the antigenic collagen fragment is a type II collagen peptide produced by cyanogen bromide cleavage. Most preferably, the fragment is the type II collagen peptide known to those skilled in the art as CB11.

The method may further include one or more additional inner ear antigens such as the signal transduction protein Raf-1, the myelin protein P0 and β-tubulin.

The detection of antibodies specific for the antigens, or combination of antigens, in the assay indicates an initial diagnosis of Meniere's disease or is useful for monitoring the progress of the disease, such as in response to therapy.

It is therefore an object of the present invention to provide a simple, rapid, sensitive and reproducible method for detecting antibodies to antigens from the inner ear.

It is a further object of the present invention to provide a method for the diagnosis and monitoring of Meniere's disease.

It is a further object of the present invention to provide a sensitive blood test for the detection of Meniere's disease in the early stages of the disease.

It is a further object of the present invention to provide an assay that can distinguish Meniere's disease from other autoimmune ear diseases.

It is a further object of the present invention to provide an assay that can monitor the progression of Meniere's disease or the effects of treatment for Meniere's disease.

These and other objects of the present invention will become apparent after reading the following detailed description of the disclosed embodiments and the appended claims.

### DETAILED DESCRIPTION OF THE INVENTION

Methods for diagnosing or monitoring autoimmune inner ear diseases such as Meniere's disease are provided. In accordance with the method, a sample is combined with one or more antigens and the binding of antibodies in the sample with one or more of the antigens is detected or measured using methods well known to those skilled in the art, such as an immunoassay.

Definitions

The terms “a”, “an” and “the” as used herein are defined to mean “one or more” and include the plural unless the context is inappropriate.

The terms “detecting” or “detected” as used herein mean using known techniques for detection of biologic molecules such as immunochemical or histological methods and refer to qualitatively or quantitatively determining the presence or concentration of the biomolecule under investigation.

By “isolated” is meant a biological molecule free from at least some of the components with which it naturally occurs.

As used herein, the term “soluble” means partially or completely dissolved in an aqueous solution.

As used herein, the term “immune sample” refers to samples having antibodies that interact specifically with one or more of the target antigens.

As used herein, the term “membranous structures” refers to the basilar membrane, organ of Corti, stria vascularis, spiral ligament and vestibular epithelium of the inner ear.

The term “antigenic variant” refers to a protein or peptide having an amino acid sequence different from the protein or peptide to which it is compared, but having similar immunologic characteristics such as the ability to bind to one or more antibodies that bind to the protein or peptide to which it is compared.

Antigen

The antigen used in the methods described herein is an inner ear collagen protein or peptide or a combination of inner ear collagen proteins, peptides or both. The antigen may be isolated or purified. The preferred collagen proteins are type II, type IX, or type XI collagen proteins. The peptide is preferably an antigenic collagen fragment. More preferably, the peptide is a collagen peptide produced by cyanogen bromide cleavage as described by Miller, Biochem. 10:3030-3035 (1971). Most preferably, the fragment is the type II collagen peptide produced by cyanogen bromide cleavage and known to those skilled in the art as CB11.

The antigen may be from any species that contains inner ear collagen proteins including, but not limited to, human, bovine, rodent or poultry.

The method may further include one or more additional inner ear antigens, preferably from the membranous structures of the inner ear, such as the signal transduction protein Raf-1, the myelin protein P0 and β-tubulin.

Myelin protein P0 is a 30 kD protein derived from acoustic nerve and spiral ganglion.

Raf-1 is a protein is highly conserved in mouse, rat, chicken, Xenopus laevis, D. melanogaster and C elegans. Raf is a serine/threonine specific protein kinase (PK) which functions in one or more signal transduction pathways between cell membrane and nucleus. The Raf-1 protein is a 74 kD protein having three conserved regions (CR). The CR1 region (approximately 53 to 200 amino acids) and CR2 region (a short sequence in the N terminal half) are considered to be critical for regulation of Raf-1 activity. C-raf is a 28 kD inner ear autoantigen as described is U.S. Pat. No. 5,885,783.

Beta-tubulin is a microtubule molecule also found in the inner ear tissue. It is a 52-kD protein.

Assay Method

The assay methods described herein determine the presence of autoimmune inner ear disease antibodies in a biological sample by detecting the binding of the antibodies to specific antigens. The antigens utilized in the assay are described above.

The binding of antibody to antigen forms an antibody-antigen complex that is detectable using various techniques. For example, the complex may be detected using a labeled antibody that binds to either the antibody or to the specific antigen. This antibody may be labeled directly with a detectable label for identification and quantitation. Labels for use in immunoassays are generally known to those skilled in the art and include enzymes, radioisotopes, and fluorescent, luminescent and chromogenic substances including colored particles such as colloidal gold and latex beads.

Alternatively, the antibody may be labeled indirectly by reaction with labeled substances that have an affinity for immunoglobulin, such as protein A or G or second antibodies. The antibody may be conjugated with a second substance and detected with a labeled third substance having an affinity for the second substance conjugated to the antibody. For example, the antibody may be conjugated to biotin and the antibody-biotin conjugate detected using labeled avidin or streptavidin. Similarly, the antibody may be conjugated to a hapten and the antibody-hapten conjugate detected using labeled anti-hapten antibody. These and other methods of labeling antibodies and assay conjugates are well known to those skilled in the art.

The immunoassay is useful for detecting the presence or amount of autoimmune antibodies in a variety of biological samples, particularly immune samples and most preferably aqueous samples such as blood, plasma, sera, cerebrospinal fluid, and the like.

The antigens described herein may be employed in any heterogeneous or homogeneous, sandwich or competitive immunoassay for the detection of autoimmune inner ear autoantibodies. Either the antigen is labeled with a detectable label or coupled to a solid phase. Methods for coupling antigens to solid phases are well known to those skilled in the art. In accordance with the immunoassay method, the sample containing the analyte is reacted with the antigen for a sufficient amount of time under conditions that promote the binding of antigen to antibody in the sample. It will be understood by those skilled in the art that the immunoassay reagents and sample may be reacted in different combinations and orders. A physical means is employed to separate reagents bound to the solid phase from unbound reagents such as filtration of particles, decantation of reaction solutions from coated tubes or wells, magnetic separation, capillary action, and other means known to those skilled in the art. It will also be understood that a separate washing of the solid phase may be included in the method.

The concentration of autoimmune antibody in the sample is determined either by comparing the intensity of the color produced by the sample to a color card or by using a reflectometer.

The resulting reaction mixture, or combination of antigen and sample, is prepared in a solution that optimizes antibody-antigen binding kinetics. An appropriate solution is an aqueous solution or buffer. The solution is preferably provided under conditions that will promote specific binding, minimize nonspecific binding, solubilize analyte, stabilize and preserve reagent reactivity, and may contain buffers, detergents, solvents, salts, chelators, proteins, polymers, carbohydrates, sugars, and other substances known to those skilled in the art.

The reaction mixture solution is reacted for a sufficient amount of time to allow the antibody to react and bind to the antigen to form an antibody-antigen complex. The shortest amount of reaction time that results in binding is desired to minimize the time required to complete the assay. A reaction time of less than five minutes is preferred. Most preferably, the reaction time is less than three minutes. By optimizing the reagents, binding may be substantially completed as the reagents are combined.

The reaction is performed at any temperature at which the reagents do not degrade or become inactivated. A temperature between approximately 4° C. and 37° C. is preferred. The most preferred reaction temperature is ambient or room temperature (approximately 25° C.).

The assay is preferably an immunoassay such as, but not limited to, an ELISA, a Western blot assay, a competitive binding assay, a particle based immunoassay, a dual particle competitive immunoassay, and any other immunoassay methods known to those skilled in the art.

For example, in a conventional immunoassay, such as an ELISA, an inert solid-phase material, usually a plastic microtiter plate, is contacted with a solution containing the target antigen so that the target antigen binds to, or coats, the solid phase material. The bound target antigen is then contacted with a sample obtained from an individual having symptoms of inner ear disease, which may or which may not contain an antibody immunoreative with the antigen. Unbound antibody is removed, and the amount of reacted antibody is detected or quantitated using any of a number of detection devices known to those skilled in the art. For example, the bound antibody may be detected with a second antibody to which has been attached a detectable label such as an enzyme, radioisotope or fluorescent molecule.

The concentration of target antigen for use in the present invention can range between approximately 1 μg/ml and 100 g/ml. The more preferable range is between approximately 3 μg/ml and 50 μg/ml. The most preferable range is between approximately 5 μg/ml and 30 μg/ml. The target antigen is dissolved in an aqueous solution and can be applied to an inert solid-phase support material by dipping, soaking, coating, spotting, spraying, blotting or other convenient means. Preferred methods include coating, spotting, spraying and blotting. More preferred methods include coating and blotting. For example, in an ELISA, a preferred volume for coating is between about 10 μl/well and 200 μl/well. A more preferred volume for coating is between about 30 μl/well and 150 μl/well. A most preferred volume for coating is between about 50 μl/well and 100 μl/well. Determination of the amount of target antigen to be used for each method of application is well within the knowledge of one skilled in the art. For example, a standard target antigen-antibody assay combination can be used to determine the amount of target antigen to be applied to the inert solid-phase support material.

The solvent for use in the assay can be any solvent that can solubilize the antibody, and that is sufficiently miscible with water to be completely removed by subsequent thorough rinsing with an aqueous solution. Such solvents include, but are not limited to phosphate buffered saline (PBS), tris(hydroxymethyl)amino methane (TRIS), N-2-hydroxyethylpeperazine-N′-2-ethanesulfonic acid (HEPES), citric acid-phosphate buffer and carbonate buffer. Such aqueous buffers and their appropriate pHs are well known to those skilled in the art. Mixtures of solvents may also be used. Preferred solvents include 0.1 M carbonate buffer, pH 9.0, and citric acid-phosphate buffer, pH 5.0. These solvents may contain other chemicals including, but not limited to, SDS, Tween-20. bromphenol blue, glycerol and dithiothreitol.

The solid phase, or inert solid-phase support material, for use in the assay can be in the form of, but is not limited to, a membrane, a bead, a microtiter plate or any other solid-phase support form known to those skilled in the art. Preferred forms include a membrane strip, a membrane well microtiter plate and a plastic well microtiter plate. More preferred forms include a membrane strip and a plastic well microtiter plate. A most preferred form is a plastic well microtiter plate. In addition, the inert solid-phase support material can be placed into a holder, including but not limited to, a membrane sheet holder, a dot-blot apparatus, a microtiter plate, a column, and a filter. Preferred holders include a membrane sheet holder, a dot-blot apparatus and a microtiter plate.

The blocking buffers for use in the present invention to prevent non-specific binding can be any suitable blocking buffer including, but not limited to, goat serum, fetal calf serum, gelatin, low fat milk, and Tween-20 at various dilutions in an aqueous solution.

The washing solution for use in the present invention can be any suitable aqueous buffer including, but not limited to, phosphate buffered saline (PBS), tris(hydroxymethyl)amino methane (TRIS) and N-2-hydroxy-ethylpeperazine-N′-2-ethanesulfonic acid (HEPES). Such aqueous buffers and their appropriate pHs are well known to those skilled in the art.

Any convenient indicator method can be used to detect binding of antibody to the target antigen. Such methods include, but are not limited to, the use of enzymes, enzyme cofactors, enzyme effectors, chromogenic substances, fluorogenic substances, chemiluminescent substances, bioluminescent substances, and labeled antibodies. Preferred indicator methods are the peroxidase-labeled antibody method and the alkaline phosphatase-labeled antibody method.

For example, the assay may be a “sandwich immunoassay”. In this assay, a solid phase substance coated with the target antigen is combined in a solution with a sample containing the antibody and reacted for a sufficient amount of time to allow the target antigen and the antibody to interact. In the detection step, a detectable substance, such as a colored bead, coated with a substance that binds readily to the antibody, such as protein A or protein G, a second antibody reactive with the antibody, or a small synthetic affinity ligand is added to the suspension. The detectable substance binds to the antibody complexed to the target antigen coated solid phase.

The complex is detected either visually with the naked eye or using a conventional detector, such as a colorimeter or reflectometer, well known to those skilled in the art. In this sandwich immunoassay, the detection of signal indicates the presence of autoimmune inner ear disease antibodies in the sample.

Assay Kit

An assay kit for detecting autoimmune inner ear disease antibodies in a biological sample is also provided herein. The kit contains one or more of the antigens described above and reagents for performing an assay to detect or measure antibodies specific for the antigens in a biological sample.

Preferably, the reagents, including the antigen are dried or lyophilized. Addition of aqueous sample to the components of the kit results in solubilization of the dry reagent and antigen, causing them to react.

For example, the kit may contain an inert solid-phase support material having target antigen immobilized thereon and may further contain reagents and a holder for the inert solid-phase support material.

The kit may additionally contain equipment for safely containing the samples, a vessel for containing the reagents, a timing means, and a calorimeter, reflectometer, or standard against which a color change may be measured.

The methods described above will be further understood with reference to the following examples, which are in no way intended to limit the scope of the present invention.

### EXAMPLE 1

### Detection of Autoimmune Inner Ear Antigens in Meniere 's Disease Patient Sera by ELISA

This example examines the presence of antibodies in sera of Meniere's disease patients against the following eight antigens by Enzyme Linked Immunosorbent Assay (ELISA): chicken type II collagen and bovine type II collagen and their cyanogens bromide cleaved peptides (CB11), type IX collagen type XI collagen, C-raf and 13-tubulin.

Materials and Methods

Sera from 108 Meniere's disease patients were obtained from a clinic. The diagnosis of Meniere's disease in these patients was based on the criteria as described by Pearson et al., Otolaryngol. Head Neck Surg. 93:579-581 (1985). Sera from 28 healthy normal controls were also obtained.

Type II collagens and their CB-II peptide and type IX and XI collagens were obtained as described by Terato, et al., J Exp. Med. 162:637-646 (1985), Miller, Biochem. 10:3030-3035 (1971), Reese and Mayne, Biochem. 20:5443-5448 (1981), Tomoda et al., in 2^{ND} INTL. SYMPOSIUM ON MENIERE'S DISEASE, Cambridge, Mass., JB Nadal, ed., Kugler & Ghedini Pub., Amsterdam, Berkley, Milan. 165-172 (1989). C-raf was obtained as described in U.S. Pat. No. 5,885,783, which is incorporated by reference herein. Beta-tubulin was obtained commercially (ICN Biomedicals Inc., Costa Mesa, Calif.).

Determination of Antibodies to Eight Antigens from Meniere's Patients by ELISA.

The specific antibodies from disease patients and 28 control sera were examined by an ELISA as follows.

Fifty microliters of eight antigens (4-5 μg/ml were dispensed in each well of a polystyrene microtiter plate and incubated overnight at 4° C. The plates were washed with 0.05% PBS-Tween buffer and incubated overnight with heavy-chain specific anti-human IgG antibodies at 4° C. The plates were washed five times before the addition of a citric acid-phosphate buffer (pH, 5.0) containing 0.15 mg/ml of o-phenylenediamine. The color was developed at room temperature and the reaction was stopped by 2.5 M sulfuric acid. The color was measured at 492 nm.

Results

The results of the ELISAs are summarized in Table Ia and Ib below.

The antibody against type II collagens (either bovine or chicken) was present about 41% to 44% of Meniere's disease patient sera.

The 38% to 41% of Meniere's disease patients' sera reacted to either of the CB11 peptide chicken or bovine type II collagens.

Type IX collagen showed 38% of binding activity with Meniere's sera and 42% with type XI collagens.

C-raf and β-tubulin each showed 55% and 61% of binding activity respectfully.

When the type II collagens and their CB11 peptide are all combined, binding activities were noted in 67% of Meniere's disease patient's sera (72 out of 108 patients). The combined type IX and XI collagen binding activities were 55% (59 out of 108). The binding activities with the combination of chicken and bovine collagen type II, their CB11 fragments, collagen IX and XI were 77% (83 out of 108).

The combination of C-raf and 13-tubulin binding activities were 80% (86 out of 108). When all eight antigens were added, 91% of sera showed binding activities to one or more of these eight antigens.

Control sera showed a universally low binding activity, one out of 28 sera with CB11 or C-raf protein, 2 out of 28 with CB11 (bovine) peptide, 3 out of 28 with CB11 (chicken), one with type IX collagen and none with β-tubulin and type XI collagen.

These results suggest that multiple antigens are involved in autoimmune ear diseases such as Meniere's disease. Among those, Raf-1, P0, C11 and beta-tubulin are, thus far, the only antigens with defined molecular characteristics. Sensitivity for each antigen falls between 38% and 61% individually, 91% when all eight antigens were combined. Specificity was 79%.

These results show that 91% of Meniere's disease sera have antibody activities to one or more of these inner ear antigens and suggest that an ELISA test to these eight inner ear antigens is useful as a diagnostic tool for Meniere's disease.

TABLE Ia
Binding of Meniere's Patient Sera Antibodies to Inner Ear Antigens

| Ag | (+) | (%) | (−) | (%) | Total |
|---|---|---|---|---|---|
| Chicken CB11 | 46 | 41 | 62 | 59 | 108 |
| Bovine CB11 | 41 | 38 | 67 | 62 | 108 |
| Chicken | 44 | 41 | 64 | 59 | 108 |
| Type II collagen | | | | | |
| Bovine | 48 | 44 | 60 | 56 | 108 |
| Type II collagen | | | | | |
| Type IX collagen | 41 | 38 | 67 | 62 | 108 |
| Type XI collagen | 45 | 42 | 63 | 58 | 108 |
| C-Rat | 59 | 55 | 49 | 45 | 108 |
| Tubulin | 66 | 61 | 42 | 39 | 108 |

TABLE Ib
Binding of Meniere's Patient Sera Antibodies to
Combinations of Inner Ear Antigens

| Ag | (+) | (%) | (−) | (%) | Total |
|---|---|---|---|---|---|
| A(C CB11 + B CB11 | 72 | 62 | 36 | 33 | 108 |
| + C II + B II) | | | | | |
| IX + XI | 59 | 55 | 49 | 45 | 108 |
| A + (IX + XI) | 83 | 77 | 26 | 23 | 108 |
| C-Raf + Tubulin | 86 | 80 | 22 | 20 | 108 |
| A11 Ag | 98 | 91 | 10 | 9 | 108 |

C CB11: cyanogen bromide fragment of chicken type II collagen
B CB11: cyanogen bromide fragment of bovine type II collagen
CII: chicken type II collagen
BII: bovine type II collagen
IX: type IX collagen
XI: type XI collagen

While the foregoing specification teaches the principles of the present invention, with examples provided for the purpose of illustration, it will be understood that the practice of the invention encompasses all of the usual variations, adaptation, modification or deletions as come within the scope of the following claims and their equivalents.

All references cited herein are hereby incorporated by reference.

Modifications and variations of the present methods and kits will be obvious to those skilled in the art from the foregoing detailed description. Such modifications and variations are intended to come within the scope of the appended claims.

What is claimed is:

1. A method for detecting an autoimmune inner ear disease antibody in a sample comprising combining the sample with an antigen and detecting the binding of the antigen to the antibody, wherein the antigen is an inner ear collagen molecule.

2. The method of claim 1 wherein the inner ear collagen molecule is a collagen protein.

3. The method of claim 1 wherein the collagen is selected from the group consisting of type II collagen, type IX collagen, type XI collagen or a combination thereof.

4. The method of claim 1 wherein the inner ear collagen molecule is an antigenic inner ear collagen peptide.

5. The method of claim 4 wherein the antigenic inner ear collagen peptide is a cyanogen bromide fragment of a collagen protein.

6. The method of claim 5 wherein the collagen protein is a type II collagen protein.

7. The method of claim 4 wherein the collagen peptide is a CB11 peptide.

8. The method of claim 1 wherein the antigen further comprises a protein selected from the group consisting of the signal transduction protein Raf-1, the myelin protein P0 and β-tubulin, or a combination thereof.

9. The method of claim 1 wherein the method is an immunoassay.

10. The method of claim 1 wherein the method is an ELISA assay.

11. The method of claim 1 wherein the sample is from a patient and the detection of antibody in the sample is used to diagnosis or monitor Meniere's disease in the patient.

12. A kit for detecting an autoimmune inner ear disease antibody in a sample comprising an inner ear collagen molecule and an immunoassay reagent.

13. The kit of claim 13 wherein the inner ear collagen molecule is a collagen protein.

14. The kit of claim 13 wherein the collagen is selected from the group consisting of type II collagen, type IX collagen, type XI collagen or a combination thereof.

15. The kit of claim 13 wherein the inner ear collagen molecule is an antigenic inner ear collagen peptide.

16. The kit of claim 15 wherein the antigenic inner ear collagen peptide is a cyanogen bromide fragment of a collagen protein.

17. The kit of claim 16 wherein the collagen protein is a type II collagen protein.

18. The kit of claim 15 wherein the collagen peptide is a CB11 peptide.

19. The kit of claim 13 wherein the antigen further comprises a protein selected from the group consisting of the signal transduction protein Raf-1, the myelin protein P0 and β-tubulin, or a combination thereof.

20. The kit of claim 13 wherein the reagent is an ELISA reagent.
